(19) United States
(12) Patent Application Publication
(10) Pub. No.: US 2018/0117300 A1
(43) Pub. Date: May 3, 2018

(54) DIRECT CURRENT APPLICATOR FOR THERAPEUTIC USE, WITH A PLURALITY OF SHEET-LIKE STRUCTURES

(71) Applicant: Albrecht MOLSBERGER, Düsseldorf (DE)

(72) Inventor: Albrecht MOLSBERGER, Düsseldorf (DE)

(21) Appl. No.: 15/561,846

(22) PCT Filed: Mar. 29, 2016
(86) PCT No.: PCT/EP2016/000525

§ 371 Date: Sep. 26, 2017

(30) Foreign Application Priority Data
Mar. 28, 2015 (DE) 10 2015 004 064.2

(51) Int. Cl. A61N 1/04; A61N 1/20; A61N 1/05

(52) U.S. Cl. CPC A61N 1/0476; A61N 1/0502; A61N 1/0408; A61N 1/20

(57) ABSTRACT

The present invention relates to a direct current applicator comprising a direct current source, or a device for attachment to a direct current source, and a sheet-like first electrode and a sheet-like second electrode for connection to the direct current source, wherein the first electrode comprises a plurality of sheet-like structures, and wherein (1) each of the midpoints of the sheet-like structures of the first electrode has a distance of 15 cm or less from at least one other such midpoint, and/or (2) one of the following alternatives applies: (i) all midpoints of the sheet-like structures of the first electrode are arranged in a polygon in which the ratio of the maximum side length to the minimum side length is 3 or less, (ii) some of the midpoints of the sheet-like structures of the first electrode are arranged in such a polygon, the remainder of the midpoints are arranged outside the polygon, and, on projection in a plane which is chosen such that the sum of the squares of the distance of the polygon corners from the plane is minimal, each midpoint lying outside the polygon has at least one connection line, intersecting the polygon, to another midpoint lying outside the polygon, or (iii) some of the midpoints of the sheet-like structures of the first electrode are arranged in such a polygon, the remainder of the midpoints are arranged outside the polygon, at least one connection line of two midpoints is parallel to at least one side line of the polygon, and, on projection in said plane, no midpoint lying outside the polygon has a connection line, intersecting the polygon, to another midpoint lying outside the polygon.

The present invention relates to a device for applying direct current. The direct current applicator according to the invention can be used for the therapeutic or cosmetic treatment of the human or animal body. Furthermore, the present invention relates to the direct current applicator for specific use in certain methods for the therapeutic treatment of the human or animal body. The direct current applicator is especially suited for the treatment of pain or of an allergy. Finally, the invention also relates to a method for manufacturing the direct current applicator.

Many medically or cosmetically relevant impairments of the human or animal body are locally caused. A medically relevant impairment is present in diseases or disturbances of the functioning of the body. In these instances a therapeutic treatment is basically indicated. In the case of irritation states, which includes in the framework of the present invention relatively slight impairments or problems of the human or animal body which do not present any disease or functional disturbance and which are not worthy of therapy, at least a cosmetic, non-therapeutic treatment is often appropriate.

The present invention relates to the creation of new therapy possibilities for (primarily local) medically relevant impairments of the human or animal body as well as to the non-therapeutic, cosmetic treatment of cosmetic impairments caused by (primarily local) irritation states.

The concept therapy also comprises prophylaxis here. “Local” means that a certain area of the medically relevant impairment or of the irritation states can be determined on or in the body. This area is preferably locally circumscribed and can preferably be exactly identified. The area (e.g. pain area, area with allergy symptomatic) is associated with certain symptoms and is associated with the complaints or the impairment.

Cluster headache (Being-Horton neuralgia) is a rare disease image with extreme and unbearable headaches. They occur in violent, one-sided attacks localized in the area of the temple and the eye, in most patients always on the same side and spontaneously fade away again typically after 15 minutes up to several hours. The attacks occur periodically in groups with differently long remission phases. The cause of the cluster headache has not been explained. There are different known variants of the acute treatment (e.g. oxygen inhalation, triptans, lidocaine) and the preventive treatment (e.g. Verapamil, corticosteroids). However, in some patients none of these treatment options is adequately effective. Due to the enormous pressure of the pain of the afflicted persons, other treatment options are urgently needed.

Allergic rhinitis is an allergically caused inflammation of the nasal mucous membrane which is regularly accompanied with other diseases of the respiratory tract, for example, inflammation of the conjunctiva, inflammation of the paranasal sinuses or asthma. Allergic rhinitis comprises seasonal allergic rhinitis (hay fever) non-seasonal allergic rhinitis (house dust allergy, i.e., allergy to domestic dust mites) and occupationally caused allergic rhinitis. An allergic rhinitis adversely and severely affects the quality of life of the afflicted patent, e.g., by sleep disturbances and a reduced ability to concentrate and/or to perform, or also by the necessity of having to avoid the allergens in question. The frequency of such diseases has greatly increased in the past decades in industrial countries, which can occasionally be explained by false reactions of immunomechanisms which originally served to protect against parasites and which are used too little due to an improved hygiene. An acutely active therapy is possible; however, many even modern drugs have the disadvantage that they make one tired or have other significant side effects. A hyposensitizing takes a long time and requires a good compliance and is nevertheless not effective in all instances.

Endogenic, physiological electrical fields are known in biology. Such fields are in the range of 70 mV/mm (nerve growth in chickens), 140 mV/mm (wound healing in rats), 600 mV/mm (eye lens of vertebrates) to 1500 mV/mm (development of the neuronal tube in the amphibian Axolotl). Depending on the inner resistance of the biological tissue concerned, currents of 10-200 μA result. Endogenic electrical fields develop for a time of hours to weeks, for example, in the wound area, in the area of the active cell growth and in the cell migration and seem to be essential for the regulation of the cell behavior.

The use of exogenic, electrical fields in medicine and cosmetics is basically known. Strong fields and/or fields that change in time are regularly used here, wherein the changing in time is brought about, for example, by alternating voltage or short direct voltage impulses. These strong electrical fields which have been therapeutically used up to the present are generated, for example, by high voltages and regularly by strong currents. Alternating current devices and impulse current devices are used in this connection in order to counteract electrolytic effects on the electrodes used and in particular on body tissue.

For example, the transcutaneous electrical stimulation of nerves (TENS) is known. In it, low frequency (1-100 Hz) biphasic alternating current impulses are used to reduce pain, primarily for the short-term “electrical analgesia”. The voltage is up to 70 V at approximately 250 μs impulse time, the current strength is up to approximately 90 mA. The effect is based primarily on an increase of the central excretion of endorphins. It is unclear whether even local and longer-term effects can be additionally achieved in the tissue concerned.

Electrical acupuncture is also known. Its active mechanism aims at excreting central, pain-reducing substances, in particular enkephalins, endorphins and dynorphins.

As is described in the document US 2004/011128 A1, electrical acupuncture also uses alternating currents. In electrical acupuncture a low-frequency stimulating current is used (Akupunktur—Lehrbuch and Atlas [German=Acupuncture—Textbook and Atlas] by Stux, Stiller & Pomeranz, third edition, Springer Verlag, 1989, p. 3), in which the frequency of the electrical signal is invariable or variable (2-10,000 Hz). Relatively strong currents ranging between 2 and 15 mA are used here as in TENS. They can only be applied at this intensity in a pulsed manner with an impulse time of about 0.3-0.6 ms. In order to avoid electrolytic effects at the transition between electrode and biological tissue at these high currents, the poling is changed (alternating current). Accordingly, the electrical parameters in electrical acupuncture have in every instance a frequency and intensity (see a summary about parameters of electrical acupuncture retrievable on the website http://www.icmart.org/ and stemming from a congress of the International Council on Medical Acupuncture and Related Techniques in 1998).

In sum, the known devices for TENS or electrical acupuncture are complex as regards their switching technology and operate with high currents, short impulses and alternating current with determined frequencies. Regardless of this, the stimulation dosage is often uncontrollable.

A direct current galvanotherapy with high current strengths of 60-80 mA at a voltage of 6-35 V is known in tumor therapy. In this therapy a destruction of the tumor tissue should occur, for example by necrotization. Therefore, a destruction of tissue is not an undesired effect to be avoided here but on the contrary expressly desired. The method makes use of the heightened conductivity of tumor tissue in comparison to healthy tissue so that the current flow should selectively be concentrated in the tumor tissue and bring about the decomposition of the tumor there by electrolytic and necrotizing effects.

Direct current is also used for the transcutaneous transport of ionizable medicaments (iontophoresis). Voltages of about 36-60 and currents of about 10-30 mA are used. In order to avoid local tissue damage here and to be able to transport a high dosage of active substances of electrophoretically, large-surface skin electrodes are placed on the skin.

Microcurrent therapy is also known and widespread. In it, alternating currents and pulsed direct currents with a very low current strength of a maximum of 900 μA and a frequency of 0.1 to 999 Hz are applied by adhesive skin electrodes. The indication field is pain diseases of the movement apparatus.

Furthermore, a use of wide-surface, moist cellulose electrodes on the head skin for stimulating the central nervous system is known, for example in the case of tinnitus (transcranial direct current stimulation, tDCS). In it a current strength up to 1 mA and a voltage of 8-25 V at a constant and pulsating current are used.

It is known that the corresponding electrical fields generated by a weak direct current further the growth of vessels, among other things via the excretion of VEGF and its influence on endothelial cells. They lead to a movement and new arrangement of cellular membrane receptors, elevate the dividing rate of certain cells and accelerate the cell migration of epithelial cells. This cellular migration takes place from the anode (from the positive pole) away and to the cathode (to the negative pole). There are indications in animal experiments that the peripheral generation of nerves can be accelerated after trauma to the spinal cord, wherein the axons of the nerve cells grow toward the cathode and which must lie facing the head for about three weeks. Clinical studies on humans indicate an acceleration of the healing of wounds by electrical fields.

The present invention is based on the technical problem of making available novel means and methods with which impairments of the human or animal body can be mitigated or eliminated, preferably pains (in particular headaches such as cluster headache, migraine headache or tension headache) and/or allergies (in particular allergic rhinitis such as seasonal allergic rhinitis, domestic dust mite allergy, animal hair allergy or allergic asthma).

The previous explanations as well as a description of exemplary embodiments contained in the present text does not exclude certain embodiments or features.

The means according to the invention preferably allows a more effective, safer, reproducible, simpler, more rapid, less stressful action or one with less side effects than the means and methods of the prior art and/or allows a treatment of impairments which can still not be adequately treated or not treated at all with the means in the prior art.

The technical problem is solved according to a first aspect of the present invention by a direct current applicator comprising a direct current source or an apparatus for being connected to a direct current source and comprises a sheet-like first electrode and a sheet-like second electrode for the connection (preferably: connected) to the direct current source, wherein the first electrode comprises a plurality of sheet-like structures, and wherein (1) each of the midpoints of the sheet-like structures of the first electrode has a distance of 15 cm or less (“greatest distance”) from at least one other such midpoint and/or wherein (2) one of the following alternatives applies: (i) all midpoints of the sheet-like structures of the first electrode are arranged on a polygon (i.e. in the form of a polygon) in which the ratio of the maximum side length to the minimum side length is 3 or less, (ii) a part of the midpoints of the sheet-like structures of the first electrode is arranged in such a polygon, the remaining part of the midpoints is arranged outside of the polygon, and given a projection in a plane which is selected in such a manner that the sum of the squares of the distance of the polygon corners from the plane is minimal, each midpoint located outside of the polygon has at least one connection line intersecting the polygon to one another midpoint located outside of the polygon, or (iii) a part of the midpoints of the sheet-like structures of the first electrode is arranged in such a polygon, the remaining part of the midpoints is arranged outside of the polygon, at least one connection line of two midpoints is parallel to at least one side line of the polygon, and given a projection into this plane, no midpoint located outside of the polygon has a connection line intersecting the polygon to another midpoint located outside of the polygon.

According to the invention the “polygon” is either planar (all corner points lie in a plane) or non-planar (not all corner points lie in a plane). The polygon corners are formed by midpoints of the sheet-like structures of the first electrode. A “midpoint” of the sheet-like structure is preferably the point of concentration of the sheet-like, geometric shape of the structure. The above-described plane is selected with the known method of the smallest squares of the distance. In a planar polygon this plane is identical with the plane of the polygon. The described projection into this plane serves to describe the arrangement of the sheet-like structures and transfers their generally three-dimensional arrangement in order to simplify the description into an imaginary two-dimensional arrangement. For example, it is necessary in a triangle with the cited geometry and two points lying outside of the triangle (midpoints of the sheet-like structures of the first electrode) that when viewing vertically onto the triangle, a line connecting the two points intersects the triangle.

A “connection line” of two points (e.g. of two such midpoints) is the shortest connection of these two points. It is in a straight line which is delimited by these two points, therefore it is geometrically a stretch.

“Outside of the polygon” denotes the case that the midpoints are not a component of the polygon. In addition, such midpoints preferably do not lie within the surface surrounded by the polygon.

The term “adhesive electrode” denotes in the present case a sheet-like electrode which can be placed on the skin surface (optionally with the hair removed) and can be fixed to there (preferably using and additives such as electrode gel or electrode paste which imparts or improves the conductivity), in particular by adhering it on.

The sheet-like first electrode consists according to the invention of a plurality of sheet-like structures which can also each be designated for the purposes of the present invention as a “pad”. The term “sheet-like” preferably designates according to the invention a planar and/or uniform design of the surface, alternatively at least a design which is substantially planar and/or uniform. The term “sheet-like” preferably excludes a design of the surface as a grid, wire, small tube, small pin or comparable structures.

The concept “comprising” also includes the meaning “consisting of” and has the latter meaning in preferred embodiments except when something different obligatorily results from the context. The same applies to conceptual variants such as, for example, “comprise” and “consist of”.

The term “direct current” denotes in the present case an electrical current whose direction does not change and whose current strength averaged over time does not substantially change under uniform frame conditions. The direct current is preferably a “clean” direct current whose current strength does not change substantially or at all under uniform frame conditions. However, certain fluctuations in time are also possible, in particular an “oscillating” direct current in which the current strength oscillates periodically around the certain average value without, however, the direction of flow changing. The direct current is, therefore, in this instance preferably a direct current undulating around a previously (preferably constant) set value. The undulation takes place preferably at a frequency between 0.001 and 10 Hz, in particular between 0.01 and 1 Hz, for example 0.1 Hz. It is preferably rectangular, sawtooth and in particular sinusoidal. The deflection of the direct current here is preferably 50% of the previously adjusted value (i.e. the values move between 150% and 50% of the previously adjusted value), in particular 40%, 30%, 20%, 15%, 10% 7.5%, 5%, 2.5% or 1%.

The present invention is based on the discovery that a weak direct current can effectively improve the cited medically or cosmetically relevant impairments and complaints, in particular pains and allergies, if it acts on the body via a sheet-like electrode with several sheet-like structures (pads) in a local electrical direct current field. In particular, a surprising effectiveness is made possible by the direct current applicator according to the invention in the case of headache (e.g., cluster headache) and allergic rhinitis (e.g., seasonal allergic rhinitis). The plurality of the sheet-like structures present according to the invention allow that the area of the medically relevant impairment or of the irritation state between the sheet-like structures can be surrounded or circumscribed and in this manner a local effect of the direct current can be achieved. The arrangement in a polygon with the described geometry makes it possible to circumscribe certain body parts (e.g. the nose) in such a manner with sheet-like structures that this surprising effectiveness is achieved during the treatment. The (midpoints of the) sheet-like structures of the first electrode which optionally lie outside of the polygon make it possible to treat other positions of the body with direct current (e.g. positions close to the eyes, for example the temples) and to further increase in this manner the effectiveness of the treatment. The surprising effectiveness is also achieved if (midpoints of the) sheet-like structures are lying at the greatest distance (as defined above) relative to each other.

The publication WO 2013/175021 A1 discloses a direct current applicator which can be used with needle electrodes or sheet-like electrodes. An arrangement of sheet-like structures at a certain distance or in certain geometry is not disclosed in it.

The using of sheet-like structures makes possible the treatment which is only slightly or not at all stressful since it is not necessary, for example, to insert needles through the skin. Such a treatment can also be readily realized and makes possible, for example, a rapid and uncomplicated use at home. This is especially advantageous in the case of pain occurring in attacks and in general in the case of impairments and complaints for which it cannot be excluded that the treatment must be carried out repeatedly. The using of and therefore the side effects of pharmaceutical agents can be reduced here or even avoided.

The above-described greatest distance is preferably 14 cm, 13 cm, 12 cm, 11 cm, 10 cm, 9.5 cm, 9 cm, 8.5 cm, 8 cm, 7.5 cm, 7 cm, 6.5 cm, 6 cm, 5.5 cm, 5 cm, 4.5 cm, 4 cm, and especially 3.5 cm.

Every midpoint of the sheet-like structures of the first electrode preferably has the relevant greatest distance from at least two or in particular at least three other such midpoints.

Each midpoint of the sheet-like structures of the first electrode preferably has a distance of 0.5 cm or more from all other such midpoints (“minimum distance”). A minimum distance is preferably 1 cm, 1.5 cm, 2 cm, 2.5 cm and in particular 3 cm.

Preferred combinations of minimum distance and greatest distance are 0.5 and 15 cm, 1 and 12, cm, 1.5 and 10 cm, 2 and 8 cm, 2.5 and 6 mc, 3 and 5 cm, 3 and 4 cm as well as 3 and 3.5 cm.

In certain instances, especially when treating pain, it is occasionally preferred to apply two pads directly adjacent to one another without an intermediate space between the pads or with an intermediate space of at the most a few millimeters in order to double the amount of the current acting on the body at a certain location.

It is preferred for the above-described polygon if the ratio of the maximum side length to the minimum side length is 2.8 or less, 2.6 or less, 2.4 or less, 2.2 or less, 2 or less, 1.9 or less, 1.8 or less, 1.7 or less, 1.6 or less, 1.5 or less, 1.4 or less, 1.3 or less, 1.25 or less, and especially preferably 1.2 or less. In some instances the ratio of the maximum side length to the minimum side length can also be 1.15 or less, 1.1 or less, 1.05 or less or even 1 (all side lengths are equal).

The above-described polygon preferably comprises (at least) one axis of symmetry. In the alternative, in which only one part of the midpoints of the sheet-like structures of the first electrode are arranged in a polygon with an axis of symmetry, midpoints of the sheet-like structures of the first electrode arranged outside of the polygon are preferably arranged symmetrically to an axis of symmetry of the polygon. These features allow an especially uniform, balanced and effective treatment.

The polygon is preferably an octagon, septagon, hexagon, pentagon, quadrangle and in particular a triangle. For the instance that only a part of the midpoints of the sheet-like structures of the first electrode are arranged in the polygon, it is preferable that an even number of midpoints of the sheet-like structures of the first electrode lie outside of the polygon. Preferably, exactly 2 of the midpoints of the sheet-like structures of the first electrode lie outside of the polygon, in particular when the polygon is a triangle.

It is preferred that each of the midpoints of the sheet-like structures of the first electrode define a corner point of the polygon. In other words, it is preferably excluded that a midpoint of a sheet-like structure of the first electrode is arranged only on one side of the polygon.

In a preferred embodiment not all angles of the polygon are equal.

A direct current applicator according to the invention is preferred in which at least one connection line of two midpoints of the sheet-like structures of the first electrode is parallel to at least one connection line of two other such midpoints.

In the direct current applicator according to the invention the number of the sheet-like structure of the first electrode is preferably 3 or greater, for example 4 or 5. Independently thereof, or preferably simultaneously, a preferred number of the sheet-like structure of the first electrode is 9 or less, for example 8, 7 or 6. Preferred ranges for the number of the sheet-like structures of the first electrode are, for example, 3 to 9, 3 to 7, 5 to 7 and 3 to 5.

In the direct current applicator according to the invention the surface of one, several or in particular of all sheet-like structures of the first electrode is in the range of 1 to 10 cm2, preferably 2 to 8 cm2, 3 to 7 cm2, 3 to 5 cm2 and in particular about 5 cm2.

The surfaces of the sheet-like structures of the first electrode are preferably identical.

The second electrode preferably has a larger surface than a sheet-like structure of the first electrode or even a larger surface than the entire surface of the first electrode. Preferred magnitudes of the second electrode are 25 cm2 to 200 cm2, in particular 50 cm2 to 100 cm2.

The effects already occur when the direct current is very weak. The current strength of the direct current on the sheet-like structures of the first electrode divided by the number of these sheet-like structures is 100 to 1000 μA. In other words, the current strength per pad is preferably 100 to 1000 μA without this necessarily, however, implying an individual maintaining constant of the current strength on each individual pad. It is also already sufficient if the entire current strength on the pads is adjusted in accordance with the number of the pads, that is, for example, in the case of three pads preferably to 300 to 3000 μA. The electrical field used in accordance with the invention is preferably on the order of endogenic and physiological electrical fields. Other preferred current strengths of the direct current on the sheet-like structures of the first electrode divided by the number of these sheet-like structures are 150 to 750 μA, 200 to 500 μA, 250 to 400 μA and in particular 300 μA.

According to the invention the first electrode has a different polarity than the second electrode. The first electrode is preferably constructed as cathode.

As a result of the direct current applicator of the invention, an improvement or elimination of cosmetically or medically relevant impairments and complaints of the body is made possible. These are preferably pains and/or allergies. According to the invention the usage of exogenic pharmaceutical active substances and/or medications is reduced or completely avoided. As a result the undesired side effects of such substances occur to a lesser extent or not at all. The impairments and complaints are ameliorated permanently or at least for a long time or are eliminated of prevented. A repetition of the usage can often increase the effect to a permanent freedom from the impairments.

The use of the direct current applicator of the invention is low-risk, effective and has few or no side effects. The effect occurs rapidly and predictably. The current dose administered can be exactly controlled. In addition, it is advantageous that according to the invention even a regeneration of tissue damaged by chronic inflammations or degenerative processes is allowed. The effectiveness against pain is according to the invention significantly better than, for example, in the case of the electrical acupuncture as described above.

The direct current applicator according to the invention has in particular an anti-allergic and analgesic effect when used on the human or animal body which is advantageous, for example, when treating (in particular local) pains or allergies. For example, headaches (in particular cluster headache, migraine headache or tension headache) or allergic rhinitis (in particular seasonal allergic rhinitis, i.e. hay fever, domestic dust mite allergy, animal hair allergy or allergic asthma) can be treated.

According to the invention human or animal bodies of patients can be treated with the direct current applicator according to the invention. The concept “patient” is not to be understood as being limited to a therapeutic treatment but rather also is covered by a cosmetic treatment. Preferred patients are mammals such as horses, dogs, cats or camels and in particular humans.

In the typical treatment course at first the area affected by the pain/the allergic reaction is localized. For example, several sheet-like structures are positioned there as described above and electrically connected to each other. The sheet-like structures are connected as the first electrode (sheet-like electrode) to a pole, preferably to the negative pole of the direct current source. The other pole is connected to the second electrode, which is preferably a surface-adhered electrode in another area of the body. Such a surface electrode is preferably placed above large muscle groups or fat layers in order that individual nerves are not irritated by the surface electrode. A preferably constant current is applied for the treatment. The pain or the allergic symptoms typically become less about 2 h to 2 days after the treatment, as a rule on the following day. The therapy time of a session is between 15 and 60 minutes. As a rule 2 to 4 sessions are carried out and in some cases even more.

An effect which can be especially well reproduced can be achieved if the current strength is held constant. The current strength determines the strength of the electrical field in the tissue. Given a constant current strength (in contrast to, for example, the setting of a constant voltage) any fluctuations of the resistance cannot produce any fluctuations of the current strength and in particular no current peaks. Even interindividual differences of the resistance cannot lead to different current strengths (in contrast to the adjusting of a constant voltage). It was found that in the case of a non-constant current strength the achieved treatment results vary from case to case to a certain extent without, however, calling in question the basic success of the treatment.

The direct current applicator of the invention (as described above) preferably comprises a means for maintaining the current strength constant during the application of the direct current (especially in the case of the changing of a resistance present between the electrodes). This means is designed in particular to maintain the current strength constant during the application of the direct current when the resistance present between the electrodes changes. Without such a means the situation frequently occurs that the electrical resistance of the body tissue (for example the skin) changes during the treatment and that the current strength then fluctuates. It is a recognition of the present invention that when using a means for maintaining the current strength constant, the result can be reproduced especially well. A constant current flow is ensured by such a means even in the case of a changing resistance and as a result a steady treatment success is achieved. In addition, it was found that without a means for maintaining the current strength constant due to an individually different resistance between skin and tissue, which is usually in the range of 3 to 100 kg hΩ, different current strengths can be observed in different individuals and as a consequence the successes of the treatment vary within certain frames. When using the means for maintaining the current strength constant, the same treatment success is achieved independently of the individually different resistance between the skin and the tissue.

Maintaining the current strength constant is preferably an automated constant maintenance. Accordingly, the direct current applicator according to the invention preferably comprises an automated means for maintaining the current strength constant during the application of the direct current (in particular in the case of a changing of the resistance present between the electrodes).

The current strength applied by an individual pad of the first electrode is designated as “individual current strength”.

It is advantageous for certain applications (e.g. regarding the reproducibility of the effect, of the strength of the effect, of the rapidity of the beginning of the effect, of the time of the duration of the effect and/or of the ability to treat certain impairments or complaints) if not only the current strength of the direct current is maintained constant if necessary but rather the particular current strength during the individual application of direct current by sheet-like structures of the first electrode is maintained constant. In other words, the particular pads of the first electrode apply a constant current strength (preferably each individual one of the pads) and not only the first electrode on the whole if necessary. For such a case one or more means for maintaining constant the current strength of the direct current applied by (the) individual pads of the first electrode are provided, as a result of which a multichannel device is created. In this case each individual channel, i.e., the current strength of the individual pads of the first electrode, can be maintained constant. A direct current applicator designed in this manner therefore comprises, in other words, one or more means for the particular maintaining constant of the individual current strengths. In a preferred embodiment such a direct current applicator comprises individually adjustable and regulatable switching circuits. However, it can also be preferred to make available such a direct current applicator which is technologically as simple as possible, for which reason even a direct current applicator is preferred which comprises several pre-resistors; preferably, each pad of the first electrode is connected in an electrically conductive manner to a pre-resistor. The pre-resistors are in this case means for maintaining constant the current strength during the individual application of direct current by sheet-like structures of the first electrode. Preferred pre-resistors have a resistance of 10 kΩ to 100 kΩ 15 kΩ to 80 kΩ 20 kΩ to 60 kΩ 25 kΩ to 40 kΩ and especially 30 kΩ. The pre-resistors are preferably sufficient to make variations of the main resistor unimportant compared to the above. Unchangeable or changeable pre-resistors can be used. Pre-resistors can be combined with one or more other means described here for maintaining constant the current strength in the individual application of direct current by (the) pads of the first electrode; however, they can also be used in the absence of such other means. Pre-resistors can alternatively be combined with the means for maintaining constant the total current strength during the application of the direct current or also can be used in the absence of such a means.

The individual current strengths can be identical or different. The individual current strengths are preferably identical, in particular in the time means, or, however, the factor which results by dividing the greatest individual current strength of the smallest individual current strength, in particular in the time means, is not greater than 2, 1.9, 1.8, 1.7, 1.6, 1.5, 1.4, 1.3, 1.25, 1.2, 1.18, 1.15, 1.12, 1.1, 1.08 or 1.05. In other cases different individual current strengths can also be preferred; it is advantageous for such cases if the individual current strengths can be individually regulated.

The fluctuation of an individual current strength about its constant value is preferably maximally 50% of the constant value, advantageously maximally 40%, 30%, 25%, 20%, 15%, 12%, 10%, 9%, 8%, 7%, 6%, 5%, 4%, 3%, 2%, or 1%. This is preferably the case for all individual current strengths.

The electrical resistance R during the treatment is determined primarily by the contact of the electrodes with the skin and optionally by the direct surroundings of the sheet-like structures. The resistance frequently changes during the course of the treatment. In order to nevertheless ensure a uniform current strength I, there are, among other things, the possibilities to change the contact surface between electrodes and body tissue, for example by changing the contact pressure of the second electrode, or by changing an internal resistance of the direct current applicator of the invention. The skin resistance can also be reduced by a long moistening of the skin and/or by heating the skin (e.g., by an infrared lamp).

The direct current applicator of the invention optionally comprises a variable inner resistance for adjusting the applied current strength.

A constancy of the current strength I is preferably ensured by appropriately varying the voltage U.

As mentioned above, a preferred means of maintaining the current strength constant in the direct current applicator of the invention is designed to be automated. This is preferably a regulator which can be built up, for example, from analogous structural components or as an integrated circuit. Such a regulator preferably comprises a means for measuring the actual current strength (for example in the supply line to the first electrode), a means for determining a deviation from a predetermined theoretical current strength, and a means for adjusting a correction of the voltage U corresponding to the deviation, in particular proportional to the deviation (proportional regulator).

Direct current sources preferred by the invention are, for example, batteries. The concept “battery” comprises in the framework of the present invention in addition to batteries with a voltage of preferably 1.2 V (for example nickel-metallic hydride batteries) up to 1.5 V (for example alkali-manganese- or zinc-carbon batteries), also accumulators (alone or connected in series in a preferred construction with two, three or four) and galvanic cells. A preferred battery has a voltage of 1.2 to 1.5 V and is present alone or preferably in a construction with two, three or four batteries connected in series. Batteries are preferably used in the absence of a means for maintaining the current strength constant during the application of the direct current in order to produce an especially simple and nevertheless already effective direct current applicator according to the invention. However, batteries can also be used in combination with a means for maintaining the current strength constant during the application of the direct current. Other direct current sources preferred according to the invention are networked parts or constant current sources. An especially preferred direct current source is contained, for example, in the device distributed by the neuroConn GmbH (Ilmenau, Germany) under the designation “DC stimulator”. This device is used in the prior art for transcranial direct current stimulation (tDCS) of the brain. It comprises an automated means for maintaining the current strength constant and is offered in a kit together with two sponge electrodes for being placed on the body for transcranial direct current stimulation.

The electrical voltage when using the direct current applicator of the invention for treating the human or animal body is preferably a maximum of 24 V, 20 V, 18 V, 16 V, 14 V, 13 V, 12 V, 11 V, 10 V, 9 V, 8 V, 6 V, 4.8 V, 4.5V, 3.6 V, 3 V, 2.4 V, 1.5 V or 1.2 V. This ensures that damaging actions to the body are avoided. The direct current applicator of the invention preferably comprises a (in particular remote-controllable) means for adjusting a maximum voltage. Furthermore, it preferably comprises a (in particular remote-controllable) means for adjusting a maximum load.

Furthermore, the direct current applicator of the invention optionally comprises a means for a time control with which several time periods for applying the direct current can be predetermined. In the simplest case this is an electronically switchable interruption of the electrically conductive between cathode, direct current source and anode. The means for the time control is preferably linked to a means for adjusting a minimum time period, wherein the latter preferably allows a determining of a minimum time period of 1 s, 10 s, 1 min, 2 min, 5 min, 10 min, 20 min or 30 min. The means for the time control can preferably be remotely controlled. According to a preferred embodiment the means for the time control is furthermore linked to a (preferably remote-controllable) means for determining a maximum time period, wherein the latter preferably allows a determination of a maximum time period of 2 h, 1 h, 50 min, 40 min, 20 min, 10 min, 5 min or 2 min.

The direct current applicator of the invention preferably comprises a (in particular remote-controllable) means for running up and running down (ramping) the current strength. Such a means allows at the beginning of the treatment a rising of the current strength from zero up to the theoretical value within a pre-determinable time period (with a preferred length of 1-60 seconds, especially preferably 5-45 seconds and in particular 10-30 seconds) and at the end of the treatment of a dropping from the theoretical value to zero within a pre-determinable time period (with a preferred length of 1-60 seconds, especially preferably 5-45 seconds, more preferably 10-30 seconds and especially 15 seconds). A slow running up and running down of the current strength is advantageous because otherwise—in the case of a sudden cutting in or cutting out of the current—the treated individual experiences an unpleasant twitching or a sensation of a current shock.

The direct current applicator of the invention optionally comprises a (preferably remote-controllable) means for switching the polarity of the electrodes during a treatment. This is preferably linked with the means for the time control so that it is, for example, possible to switch the polarity every second, every 10 s, every minute, every 2 min, every five min or every 10 min.

The direct current applicator of the invention can be optionally adjusted to a test mode in which a constant voltage of approximately 1-8 V, 2-6 V or 3-5 V is applied. This can check whether the electrodes are coupled in an electrically correct manner or not or whether there is a hidden cable break in some cable. The direct current applicator of the invention preferably comprises a signal device, (for example a tone generator) which indicates when a correct current flow is produced. It can be concluded from an absence of the signal that the coupler chain has been interrupted.

A signal device can also be preferably used to indicate the beginning and/or the end of the treatment. Furthermore, a signal device can be preferably used to indicate if the current flow is interrupted during a treatment or the impedance of the patient is too high, in particular in combination with a cut-off means.

The direct current applicator of the invention is preferably adapted, as described above, to an application of pulsed direct current with a frequency of 1 Hz or lower or to an application of continuous current (i.e. of a non-pulsed direct current).

The first electrode furthermore preferably comprises, i.e. in addition to the sheet-like structures, one or more needles, preferably 3 to 12, 4 to 10, 6 to 10, 6 to 8 and especially 8. The term “needle” denotes an oblong (preferably cylindrical) body whose length is large relative to it diameter. A needle preferably has a pointed end, in particular a conically pointed one. A needle is preferably constructed in such a manner that the human or animal body is not injured during its use. The diameter of an area to be inserted is (without taking into consideration a sharpened end) preferably between 0.1 and 0.8 mm, preferably between 0.2 and 0.4 mm and especially approximately 0.3 mm, wherein an area to be inserted furthermore preferably has a sharpened end; the length of an area to be inserted is preferably between 10 and 100 mm, preferably between 20 and 50 mm and in particular approximately 30 mm. The diameter in a handle area can be, for example, approximately 1-3 mm. Preferred needles have the shape of known acupuncture needles and the following dimensions: 0.2×15 mm, 0.25×40 mm, 0.3×30 mm, 0.3×100 mm, 0.35×50 mm and 0.35×100 mm. The material of a needle is preferably metal. Preferred metals are high-grade steels, i.e. non-alloyed or allied steels with a low content of sulfur and phosphorus. Other alloy components are preferably chromium (preferably in a portion of 10.5-13 weight % or higher), nickel (preferably in a low portion, approximately maximally 10% by weight, molybdenum, titanium and/or niobium. 18/10 chromium-nickel steel or medicinal high-grade steel is preferred. Preferred steels are those which are resistant to water and to weak organic and inorganic acids. Non-rusting steels are especially preferred. Other preferred metals are silver, gold and platinum. The needles are optionally coated with silver, gold or platinum. Sintering tools, for example consisting of silver/silver chloride are also preferred.

The needle or needles optionally used adjacent to the sheet-like structures of the first electrode is/are preferably arranged outside of an area circumscribed by the sheet-like structures.

To the extent that the first electrode comprises one or more needles adjacent to the sheet-like structures, it is preferred to regulate the current strength on the needles separately from the current strength on the sheet-like structures. Then, that which was discussed above applies to the latter. The current strength of the direct current on the needles of the first electrode is then preferably 10 to 100 μA, preferably 15 to 80 μA, 20 to 60 μA, 25 to 50 μA and 25 to 40 μA, and in particular 30 μA.

An especially preferred direct current applicator of the invention is structured as follows, and therefore has a sum of features which are preferred already, each taken by itself: The first electrode consists of a plurality of sheet-like structures. The polygon is a triangle with an axis of symmetry. Two midpoints of sheet-like structures of the first electrode are optionally arranged outside of the triangle which are arranged symmetrically to the axis of symmetry of the triangle. The current strength of the direct current divided by the number of the sheet-like structures of the first electrode is 100 to 1000 μA and in particular 300 μA. The first electrode is constructed as cathode. The surface of all sheet-like structures of the first electrode is in the range of 1 to 10 cm2 and in particular 5 cm2 The direct current applicator is adapted to an application of continuous current. The direct current applicator comprises a means for automatically maintaining the current strength constant during the application of the direct current. During the treatment of cluster headaches the first electrode furthermore comprises several needles and on the other hand does not contain any during the treatment of seasonal, allergic rhinitis (hay fever).

According to a second aspect the present invention also relates to the direct current applicator of the invention (as described above, in particular in one or more of the preferred embodiments) for being used in the treatment of pain (preferably headaches, in particular cluster headache, migraine headache or tension headache) or an allergy (preferably allergic rhinitis, preferably seasonal, allergenic rhinitis, domestic dust mite allergy, animal hair allergy or allergic asthma).

In other words, subject matter of the second aspect of the present invention also comprises the using of such a direct current applicator of the invention in the treatment of pain (preferably headache, in particular cluster headache, migraine headache or tension headache) or an allergy (preferably allergic rhinitis, preferably seasonal, allergenic rhinitis, domestic dust mite allergy, animal hair allergy or allergic asthma) and the using of such a direct current applicator of the invention for manufacturing a therapeutic device for being used in the treatment of pain (preferably headache, in particular cluster headache, migraine headache or tension headache) or an allergy (preferably allergic rhinitis, preferably seasonal, allergenic rhinitis, domestic dust mite allergy, animal hair allergy or allergic asthma). In other words, the second aspect of the present invention relates to a method for the treatment of pain (preferably headache, in particular cluster headache, migraine headache or tension headache) or an allergy (preferably allergic rhinitis, preferably seasonal, allergenic rhinitis, domestic dust mite allergy, animal hair allergy or allergic asthma) of a patient who requires such a treatment, comprising allowing the direct current applicator of the invention to act on the body of the patient.

A third aspect of the present invention relates to the usage of the direct current applicator of the invention (as described above, in particular in one or more of the preferred embodiments) for the cosmetic treatment of the human or animal body.

According to a fourth aspect the present invention also relates to a method for manufacturing a direct current applicator, preferably as described above (in particular in one or more of the preferred embodiments), comprising the following steps:
(a) Making a plurality of sheet-like structures available for usage as first electrode,
(b) Making a sheet-like electrode available for usage as second electrode,
(c) Making a direct current source or a device for connection to a direct current source available,
(d) Arranging the sheet-like structures of the first electrode in such a manner that (1) each of the midpoints of the sheet-like structures of the first electrode has a distance of 15 cm or less (“greatest distance”) from at least one other such midpoint and/or that (2) one of the following alternatives applies: (i) all midpoints of the sheet-like structures of the first electrode are arranged on a polygon in which the ratio of the maximum side length to the minimum side length is 3 or less, (ii) a part of the midpoints of the sheet-like structures of the first electrode is arranged in such a polygon, the remaining part of the midpoints is arranged outside of the polygon, and given a projection in a plane which is selected in such a manner that the sum of the squares of the distance of the polygon corners from the plane is minimal, each midpoint located outside of the polygon has at least one connection line intersecting the polygon to one another midpoint located outside of the polygon, or (iii) a part of the midpoints of the sheet-like structures of the first electrode is arranged in such a polygon, the remaining part of the midpoints is arranged outside of the polygon, at least one connection line of two midpoints is parallel to at least one side line of the polygon, and given a projection into this plane, no midpoint located outside of the polygon has a connection line intersecting the polygon to another midpoint located outside of the polygon, and
(e) Optionally connecting the first and the second electrode to the direct current source.
   That which was explained above applies in a corresponding manner to preferred embodiment of the method according to the invention for manufacturing a direct current applicator.

The following concepts are used in the description of the following examples:

NSM: Treatment with direct current while maintaining the current strength constant.

VAS: Visual analog scale with 0=not complaints and 10=unbearable complaints, e.g., pain or allergic complaints.

Needles=acupuncture needles with thickness 0.25, length 40 mm, metal and allow as described above.

CK: Cluster headache

### EXAMPLE 1: PAIN

Female patient 52 years old: Very strong, chronic cluster headache (CK) on the left for 3 years. More intense during the night, up to 8 attacks per day, strength VAS 7-10, duration 20-90 minutes per attack. Treatment only still with oxygen; cortisone, triptan and lithium stopped on account of incompatibility. Pain radiates from the back of the head to the temple, for head, medial eyebrow, nasal wings. Patient is suicidal, 6 treatments with NSM stimulation. 6-12 needles inserted in the area of the painful areas. Total stimulation strength between 120 and 500 μA depending on the number of needles. Since no improvement after the fourth session, use of surface electrodes: adhesive pads approximately 2 cm²—one pad in the area of each of the temples/start of hair, one pad approximately centrally above the eyebrow, one pad approximately centrally underneath the eye. Total stimulation strength of the pads 750 μA, corresponding to 250 μA per pad. 3 needles simultaneously inserted 0.25 mm times 40 mm above the acupuncture points Bl 10, Gb 20, each stimulated with 40 μA. One day after the treatment reduction of the attack strength by 50% with unchanged frequency of attacks. Repetition of the same therapy after one week. Patient free of complaints for 8 weeks thereafter. The subsequently occurring recidivism is treated in the same manner with the same success.

### EXAMPLE 2: PAIN

Patient, 56 years old. Diagnosed CK since 2002, always right, diagnosis time 2 years, last episode 7 months ago, CK free for more than one hear, 4 weeks. Previous episode duration 3-7 months, CK 2-3/day. Duration 20-30 min with oxygen 10 liters, 90 min. Pain strength VAS 7-10. Sumatriptan tbl. Cortisone does not help, last 2008. Verapamil 480 mg/day. 2008-2010 unbearably strong pains which no longer respond to O2—suicidal. NSM stimulation 11 needles 0.25 mm, each 440 μA. Puncture at the acupuncture points Gb 21, Bl 10, Taiyang, Gb 2. Local anesthesia, in the following week attacks equally frequent. Pain strength less, no longer destroying. Repetition of this therapy at weekly intervals. After the 3d treatment again increase of the pains up to pain strength 10 (VAS). Directly after the treatment strong CK attack on the way back home—probably rebound due to irritation by needle treatment. After the fourth to the seventh treatment use of adhesive pads, two pads middle forehead (directly adjacent). Stimulation of the pads with a total of 600 μA. Additional 4 needles 0.25 back of head stimulated with 20 μA per needle. Immediate improvement with this therapy modification up to free of complaints after the seventh treatment. No rebound attacks any more after the treatments with pads.

### EXAMPLE 3: PAIN

Patient, 49 years old: Cluster for 20 years, episodes earlier 6 weeks to finally 7 months. Since 2012 without interruption CK with constant basic pain, drugs for years, otherwise attacks. Currently 560 mg Verapamil, Topiramate 100 mg, therefore pain free down to basic pain strength 2-3 (VAS). Without drugs pain up to VAS 10. 20 cigarettes/day. First NSM therapy 9×0.25 mm needles, 320 μA, 3 back of head, 6 in the pain area of the face. No improvement. Second session in the same manner Thereafter base pain elevated. Third NSM treatment: Use of two pads, one pad each on the forehead approximately centrally above the eye, one pad on each temple/beginning of hair. No use of needles. Stimulation per pad 250 μA. Improvement of the base pain in the following week. After 2 weeks new treatment with pads in the same manner includes further improvement of the base pain to strength 1. Topiramate and Verapamil were each able to be reduced by 50%.

### EXAMPLE 4: ALLERGY

Patient 35 years old: Seasonal allergy, pollen and grasses for more than 15 years. Eyes eye irritation VAS 5, nasal complaints with itching and flowing cold currently strength 3. One NSM treatment, 3 pads at the acupuncture points Ex 1 Di 20 with a total of 900 μA, therefore 300 μA per pad (current distribution on pads over pre-resistances 30 kΩ). Improvement approximately 4 hours after first treatment. Worse again after 3 days due to working in the garden. New treatment with the same pad position, now with 400 μA per pad, total current 1200 μA. New improvement one day after the treatment. Two other treatments each with a 3 day interval—thereafter complaint-free as regards the allergy.

### EXAMPLE 5: ALLERGY

Patient 58 years old. Known hay fever for more than 20 years. Received 10 acupunctures for prevention with success every 2 years. This year strong allergic reaction in the eyes VAS=7 and nose VAS=6. Cetirizine tablets and Nasonex drops (mometasone furoate). First NSM treatment with 5 pads and 1500 μA, one pad on Ex 1, and one pad each on Di 20 and one pad each on the lateral orbital margin. One day after the treatment 80% reduction of complaints. Second treatment in the identical manner Thereafter symptom-free for the entire summer.

### EXAMPLE 6: ALLERGY

Female patient 38 years old: since youth suffers allergies, early and late bloomer, nose VAS 7, eyes 8, lung problems with cough and beginning asthmatiform trouble breathing at 4—due to these symptoms the patient cannot perform her profession as a fitness trainer without drugs. In the past always Cetirizine and cortisone depot injections. A first NSM treatment with 4 pads each with 400 μA—total current strength 1600 μA. Positioning at the acupuncture points Ex 1, Di 20, Gb 1, Ren 22. After 2 sessions with an interval of 3 days the patient was free of complaints for the entire season. No other drugs taken. Patient can again work as a fitness trainer without problems.

### EXAMPLE 7: ALLERGY

Female patient: Pronounced hay fever for 15 to 20 years. hazel bloom, complaints since at the latest beginning of January to October. Therapy 20 mg Ebastin and evenings nasal spray, not satisfactory. Two NSM treatments end of April and beginning of May. Needle 0.3×30 mm on the nasal root, left and right next to the nasal root, on the lateral orbital margin, adjacent to the nasal wings at the level of the nose opening—a total of 7 needles placed corresponding to the acupuncture points extra 1, Gb 1, Bl 2 and Di 20. Stimulation per needle with 40 μA Immediate improvement by 90 to 100%, hardly any more allergy problems, sleeping without nasal spray possible already on the evening of the second day. No more drugs for the remainder of the hay fever season required. In the following January very light symptoms but not permanent. Quality of life enormously improved.

### EXAMPLE 8: ALLERGY

Patient: Allergic for 15 years to domestic dust mites, cat hair and hay fever (grasses and pollen). Cetirizine daily during the entire year. Strength of complaints in spite of drug treatment VAS nose 10, eye 8. 2 NSM treatments in October, 2014 at an interval of one week. Needle 0.3×30 mm at the nasal root, left and right adjacent to the nasal root, on the lateral orbital margin, adjacent to the nasal wings at the level of the nasal opening—a total of 7 needles placed corresponding to the acupuncture points extra 1, Gb 1, Bl 2 and Di 20. Stimulation each needle with 40 μA. Starting after the first treatment, improvement after the second treatment of the complaints of nose and eyes upon exposure to the above-cited allergens by at least 60%. The improvement is persistent. Hay fever complaints in the next season (January to July) did not occur until up to the observation time at the end of March.

1. A direct current applicator comprising a direct current source of an apparatus for connection to a direct current source and a sheet-like first structure and a sheet-like second electrode for the connection to the direct current source, wherein the first electrode comprises a plurality of sheet-like structures, characterized in that each of the midpoints of the sheet-like structures of the first electrode has an interval of 1 cm or more to all other such midpoints, and that one of the following alternatives is relevant: (i) all midpoints of the sheet-like structures of the first electrode are arranged on a polygon (i.e. in the form of a polygon) in which the ratio of the maximum side length to the minimum side length is 3 or less, (ii) a part of the midpoints of the sheet-like structures of the first electrode is arranged in such a polygon, the remaining part of the midpoints is arranged outside of the polygon, and given a projection in a plane which is selected in such a manner that the sum of the squares of the distance of the polygon corners from the plane is minimal, each midpoint located outside of the polygon has at least one connection line intersecting the polygon to one another midpoint located outside of the polygon, or (iii) a part of the midpoints of the sheet-like structures of the first electrode is arranged in such a polygon, the remaining part of the midpoints is arranged outside of the polygon, at least one connection line of two midpoints is parallel to at least one side line of the polygon, and given a projection into this plane, no midpoint located outside of the polygon has a connection line intersecting the polygon to another midpoint located outside of the polygon.

2. The direct current applicator according to claim 1, wherein each of the midpoints of the sheet-like structures of the first electrode has an interval of 15 cm or less to at least one other such midpoint.

3. The direct current applicator according to one of the previous claims, wherein the polygon has an axis of symmetry.

4. The direct current applicator according to claim 3, wherein midpoints of the sheet-like structures of the first electrode, which midpoints are arranged outside of the polygon, are arranged symmetrically to an axis of symmetry of the polygon.

5. The direct current applicator according to claim 3 or 4, wherein the polygon is a triangle and optionally exactly two of the midpoints of the sheet-like structures of the first electrode lie outside of the polygon.

6. The direct current applicator according to one of the previous claims, wherein at least one connection line of two midpoints of the sheet-like structures of the first electrode is parallel to at least one connection line of two other such midpoints.

7. The direct current applicator according to one of the previous claims, wherein the current strength of the direct current on the sheet-like structures of the first electrode divided by the number of these sheet-like structures is 100 to 1000 μA.

8. The direct current applicator according to one of the previous claims, wherein the first electrode is constructed as cathode.

9. The direct current applicator according to one of the previous claims, wherein the surface of one, several or all sheet-like structures of the first electrode is in the range of 1 to 10 cm².

10. The direct current applicator according to one of the previous claims, wherein the number of the sheet-like structures of the first electrode is 3 or greater and or the number of the sheet-like structures of the first electrode is 9 or less.

11. The direct current applicator according to one of the previous claims, which is adapted to an application of pulsed direct current with a frequency of 1 Hz or less or to an application of continuous current.

12. The direct current applicator according to one of the previous claims, wherein the first electrode furthermore comprises one or several needles.

13. The direct current applicator according to one of the previous claims, furthermore comprising a means for maintaining the current strength constant during the application of the direct current, wherein the maintaining constant is preferably an automated constant maintaining.

14. The direct current applicator according to one of the previous claims, for usage in the treatment of pain or of an allergy.

15. The direct current applicator for being used according to claim 14, wherein the pain is headache, in particular cluster headache, migraine headache or tension headache and/or the allergy is an allergic rhinitis, in particular seasonal allergic rhinitis, domestic dust mite allergy, animal hair allergy or allergic asthma.

16. The using of a direct current applicator according to one of claims 1 to 13 for the cosmetic treatment of the human or animal body.

17. A method for manufacturing a direct current applicator, preferably as described in one of the claims 1 to 13, comprising the following steps:
(a) Making a plurality of sheet-like structures available for usage as first electrode,
(b) Making a sheet-like electrode available for usage as second electrode,
(c) Making a direct current source or a device for connection to a direct current source available,
(d) Arranging the sheet-like structures of the first electrode in such a manner that each of the midpoints of the sheet-like structures of the first electrode has a distance of 1 cm or more from all other such midpoints and that one of the following alternatives applies: (i) all midpoints of the sheet-like structures of the first electrode are arranged on a polygon in which the ratio of the maximum side length to the minimum side length is 3 or less, (ii) a part of the midpoints of the sheet-like structures of the first electrode is arranged in such a polygon, the remaining part of the midpoints is arranged outside of the polygon, and given a projection in a plane which is selected in such a manner that the sum of the squares of the distance of the polygon corners from the plane is minimal, each midpoint located outside of the polygon has at least one connection line intersecting the polygon to one another midpoint located outside of the polygon, or (iii) a part of the midpoints of the sheet-like structures of the first electrode is arranged in such a polygon, the remaining part of the midpoints is arranged outside of the polygon, at least one connection line of two midpoints is parallel to at least one side line of the polygon, and given a projection into this plane, no midpoint located outside of the polygon has a connection line intersecting the polygon to another midpoint located outside of the polygon, and
(e) Optionally connecting the first and the second electrode to the direct current source.
